(19) United States
(12) Patent Application Publication
(10) Pub. No.: US 2005/0228409 A1
(43) Pub. Date: Oct. 13, 2005

(54) Device for anastomosis

(75) Inventor: Gioacchino Coppi, Modena (IT)

Correspondence Address: YOUNG & THOMPSON, 745 SOUTH 23RD STREET, 2ND FLOOR, ARLINGTON, VA, 22202, US

(73) Assignee: G.A.M.A.-H.S. S.R.L., BOLOGNA (IT)

(21) Appl. No.: 10/507,348

(22) PCT Filed: Nov. 14, 2003
(86) PCT No.: PCT/IT03/00741

§ 371 Date: Sep. 13, 2004

(30) Foreign Application Priority Data
Nov. 21, 2002 (IT) M02002A000337

(51) Int. Cl. A61B 17/08

(52) U.S. Cl. 606/153

(57) ABSTRACT

The device includes a tubular element (2) having a first end (2a) and a second end (2b). The tubular element (2) bears, in proximity of at least one of the first end (2a) and the second end (2b), a plurality of slender elements (3) which project in an outwards direction.

### TECHNICAL FIELD

The invention relates to a device for anastomosis.

In particular, the invention is usefully applied in the treatment of thoracic-abdominal aortic aneurysm, especially in the field of aortic prostheses connecting healthy aortic tracts.

### BACKGROUND ART

Aortic aneurysm refers to a progressive relaxing of the walls of the aorta, which leads to a dilation of the aorta with possibility of rupture and consequent serious internal haemhorraging.

The classic and most-applied art for treatment of this pathology is a surgical operation in which a tract of damaged aorta is sectioned and substituted by a tubular prosthesis made of a biocompatible material, such as Dacron or PTFE, which is then sutured to healthy tracts of the aorta using, as a rule, polypropylene wire.

Although treatment of the aneurysm using the prior art is one of the greatest conquests in the history of surgery, and leads to a practically complete recovery on the part of the patient, the surgical intervention is highly invasive and involves a not-irrelevant number of complications.

The application of the aortic prosthesis requires a large laparotomy and considerable surgical dissection. During the sectioning operations of the tract suffering from aneurysm and the consequent suturing of the prosthesis, aortic circulation must be stopped by means of ligation, performed upstream of the dilated tract.

The suture operation (prosthesis to aorta) is known as anastomosis and is carried out according to a technical principle using diagonal stitching. It involves ligating the aorta upstream of the tract to be anastomosed and suturing the prosthesis by stitching large-denier wire at intervals of about 2 mm. This technique requires ligations to be in place for the entire duration of the suture and can also require the use of various aids to improve the anastomosis, such as extra stitching or Dacron collars superposed on the suture line.

The duration of the necessary haemostasis, i.e. the period in which blood circulation is interrupted, is proportional to the level of difficulty of the aortic sectioning operations and suture of the prosthesis, and is therefore quite long. The prolonged lack of blood flow to the organs situated downstream of the operated aortic tract (suffering from aneurysm) can lead to grave complications, including sudden death, kidney failure and respiratory failure, and paraplegia due to medullar ischemia.

The main aim of the present invention is to provide an instrument which obviates the above-described problems, especially by considerably simplifying the anastomosis operations between the prosthesis and the aorta, i.e. the suture operations between the prosthesis and the aorta.

A further aim of the present invention is to reduce the invasiveness of the surgical operation for treatment of aortic aneurysm, reducing the size of the laparotomy needed for performing the anastomosis between prosthesis and aorta.

A further aim of the present invention is to simplify anastomosis between prosthesis and aorta, limiting the duration of haemostasis upstream of the aortic tract suffering from aneurysm.

### DISCLOSURE OF INVENTION

Further characteristics and advantages of the present invention will better emerge from the detailed description that follows, of a preferred but non-exclusive embodiment of the invention, illustrated purely by way of a non-limiting example in the accompanying figures of the drawings, in which:

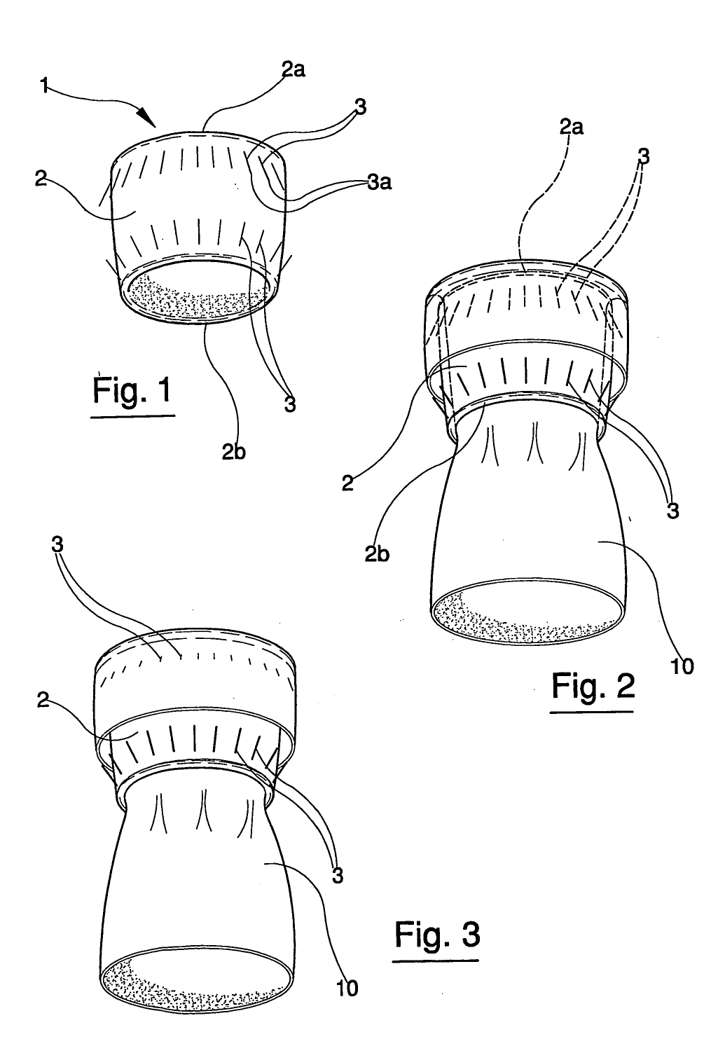

FIG. 1 is a perspective view of the device according to the present invention;

FIG. 2 shows a first stage of use of the device of FIG. 1;

FIG. 3 shows a second stage of use of the device of FIG. 1;

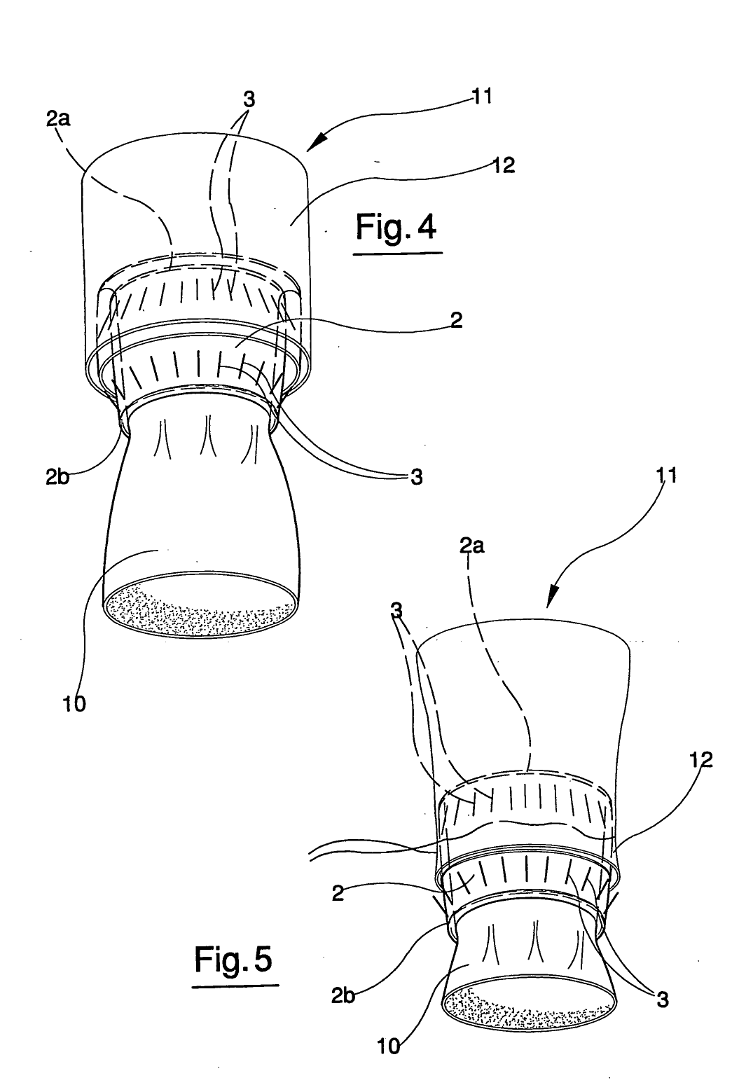

FIG. 4 shows a third stage of use of the device of FIG. 1;

FIG. 5 shows a fourth stage of use of the device of FIG. 1.

With reference to the figures of the drawings, 1 denotes in its entirety a device for anastomosis according to the invention. It comprises a tubular element 2 which exhibits a first end 2a and a second end 2b and bears, in proximity of at least one of the first and second ends 2a and 2b, a plurality of slender elements 3 which project outwardly. The slender elements 3, as shown in the embodiment of FIG. 1, are arranged in proximity of the first end 2a and exhibit a free end 3a facing towards the second end 2b. In a second embodiment, shown in FIG. 2, the device of the invention exhibits a plurality of slender elements 3 projecting outwardly in proximity of the first end 2a and a plurality of slender elements 3 projecting outwardly in proximity of the second end 2b. The slender elements 3 exhibit a free end 3a facing towards the opposite end with respect to the end at which they are located. The slender elements 3 arranged in proximity of the first end 2a can be termed proximal slender elements 3, while the slender elements arranged in proximity of the second end 2b can be termed distal slender elements. The tubular element 2 exhibits, in longitudinal section, an approximately truncoconical profile, with a decreasing transversal section in the direction going from the first end 2a to the second end 2b.

The slender elements 3 are arranged along a first circumference close to the first end 2a and along a second circumference close to the second end 2b. The slender elements 3 arranged in proximity of the first end 2a are reciprocally distanced at a closer step than the slender elements 3 arranged in proximity of the second end 2b, and are longer and more prominent with respect to the slender elements 3 arranged in proximity of the second end 2b.

The device can be applied according to the following stages.

As shown in FIG. 2, a prosthesis 10 is passed into the tubular element 2 and is externally folded over the first end 2a. The segment of prosthesis which has been folded over the first end 2a is fastened on the proximal slender elements 3, so that the slender elements 3 penetrate completely in and through the wall of the prosthesis 10, exiting therefrom by the free ends 3a thereof. As the prosthesis 10 used is not circumferentially elastic, on being folded outwardly it might ruffle and bend; to avoid this eventuality the folded tract of prosthesis 10 can be slit in a longitudinal direction in order to give a minimum level of circumferential deformability to the prosthesis 10. Alternatively a segment of the prosthesis 10 could be connected to the tubular element 2 in the above-described way, in which the tract to be folded over the tubular element 2 exhibits an increased diameter which is calculated to suit the deformation it will undergo. The prosthetic segment thus exhibits an end which is folded externally over the tubular element 2 and a free end projecting from the tubular element 2 which is connected to a normal aortic prosthesis.

The prosthesis 10, connected in one of the above-described ways to the device of the invention, can be sutured to the aorta, denoted by number 11 in FIGS. 3 and 4, in a very simple way. Once the aorta has been ligated and the dilated tract sectioned, a large-step in-and-out suture is performed at the neck of the section 12 upstream of the removed part of aorta. The prosthesis 10 is then inserted into the neck 12 of the aorta 11 and the suture is pulled tight on the portion of tubular element 2 comprised between the two ends 2a and 2b. The free ends 3a of the proximal slender elements 3 penetrate into the aortic wall, preventing any tendency of the prosthesis 10 to displace in a downwards direction.

The device for anastomosis of the present invention offers important advantages. Firstly, the anastomosis operations between the prosthesis and aorta are extremely simple and rapid, as the anastomosis is limited to performance of the straight in-and-out suture on the proximal neck of the aorta. Further, as the suture is performed using large-step stitches, the risk of ischemia of the aorta wall is limited, and as a consequence so is detachment of the prosthesis.

Secondly, the rapidity of the operations required enables a limitation of the time needed for haemostasis performed upstream of the tract comprising the aneurysm, considerably lowering the risk of complication due to lack of blood flow to the organs located downstream of the point where the aorta is ligated.

Thirdly, the surgical operation required for treatment of the aneurysm is less invasive. As only a simple straight in-and-out suture is required, the length of the laparotomy needed is considerably smaller than what is necessary for a surgical intervention made according to the prior art.

1). A device for anastomosis, wherein the device comprises a tubular element (2) having a first end (2a) and a second end (2b) and bears a plurality of outwardly-projecting slender elements (3) arranged in proximity of at least one of the first end (2a) and the second end (2b).

2). The device of claim 1, wherein the slender elements (3) are arranged in proximity of the first end (2a) and exhibit a free end (3a) facing towards the second end (2b).

3). The device of claim 1, wherein the device exhibits a plurality of slender elements (3) projecting externally in proximity of the first end (2a) and a plurality of slender elements (3) projecting externally in proximity of the second end (2b).

4). The device of claim 3, wherein the slender elements (3) exhibit a free end (3a) facing towards an opposite end from an end at which the slender elements (3) are arranged.

5). The device of claim 2, wherein the tubular element (2) exhibits a longitudinal profile section which is truncoconical and a transversal section which decreases in a direction going from the first end (2a) to the second end (2b).

6). The device of claim 5, wherein the slender elements (3) are arranged along a first circumference of the device which is proximal to the first end (2a) and along a second circumference thereof which is proximal to the second end (2b).

7). The device of claim 6, wherein the slender elements (3) arranged in proximity of the first end (2a) are reciprocally distanced at a smaller step with respect to a step at which the slender elements (3) arranged in proximity of the second end (2b) are reciprocally distanced.

8). The device of claim 7, wherein the slender elements (3) arranged in proximity of the first end (2a) are longer and more prominent than the slender elements (3) arranged in proximity of the second end (2b).

9). The device of claim 4, wherein the tubular element (2) exhibits a longitudinal profile section which is truncoconical and a transversal section which decreases in a direction going from the first end (2a) to the second end (2b).
